(19) United States
(12) Patent Application Publication
(10) Pub. No.: US 2005/0133045 A1
(43) Pub. Date: Jun. 23, 2005

(54) Natural rubber articles for treating symptoms caused by electro-magnetic pollution

(76) Inventor: Giovanni Giannotti, Varese (IT)

Correspondence Address: James V. Costigan;Hedman & Costigan, P.C., 1185 Avenue of the Americas, New York, NY, 10036-2646, US

(21) Appl. No.: 11/017,536

(22) Filed: Dec. 20, 2004

(30) Foreign Application Priority Data
Dec. 22, 2003 (IT) MI2003A002564

(51) Int. Cl. A61N 1/00; A61B 19/00

(52) U.S. Cl. 128/897; 128/898

(57) ABSTRACT

The present invention relates the use of natural rubber for making articles of manufactures such as footwear, footwear soles, seats, desks, beds or any other type of furniture articles or parts thereof, to prevent and/or treat symptoms caused by electromagnetic pollution such as: sleep disorders, weariness, difficult concentration, irritability, anxiety, memory disorders, cephalea associated with a flushing of the face, female hormonal disorders such as amenorrhea, skin symptoms such as face smarting, itching, acne eruptions, redness, erythema, muscle and joint pain, eye burning, palpitations, tendinitis and so on.

### BACKGROUND OF THE INVENTION

The present invention relates to the use in the medical field of natural rubber. More specifically, the invention relates to a method for treating symptoms caused by electromagnetic pollution.

As is known, for “electromagnetic sensitiveness” it is intended the capability of the human body to sense an exposure to electric and electromagnetic fields, without generating pathologic symptoms.

The so-called “electromagnetic hypersensitiveness” (EHS) is that feature, of electrosensitive persons, of generating pathologic symptoms after an exposure to electromagnetic fields.

More specifically, the electromagnetic field exposure symptoms, which have been conventionally found in persons having an electromagnetic hypersensitiveness are the following: sleep disorders, weariness, difficult concentration, irritability, anxiety, memory disorders, cephalea associated with a flushing of the face, female hormonal disorders such as amenorrhea, skin symptoms such as face smarting, itching, acne eruptions, redness, erythema, muscle and joint pain, eye burning, palpitations, tendinitis. This, as stated, is caused by the electromagnetic pollution or electrosmog.

Electromagnetic pollution is, by definition, a long duration exposure to electromagnetic fields (EMF) and non ionizing electromagnetic radiations (NEMR).

Natural electromagnetic fields, such as those generated by the earth (the earth magnetic field) and by the human body, the nervous system of which transmits information between the brain and other parts of the body, through electric impulses, are well known. These electromagnetic fields have a very low intensity, to which our body is accustomed, and without which a person could not survive. The progress of science has introduced into our daily life, both in our homes and outside environment, electric instruments and devices such as, for example: TV's, computer screens, hair driers, cellular and cordless phones, electric blankets and so on, in addition to high voltage cables, electric lines for trains and so on, installed near dwelling places. Since any electric current generates an electromagnetic field, each person is constantly subjected to electrosmog symptoms, as above disclosed. Moreover, in addition to electromagnetic fields, non ionizing electromagnetic radiations also exist, which are mainly emitted by transmitting antennas, radar systems and microwave ovens and which can also cause the above disclosed symptoms.

The above electromagnetic fields are conventionally quantified by frequency parameters, as measured in hertz (Hz) or kilohertz (kHz), and intensity or strength parameters, as measured in gausses or milligausses (mG). Frequencies which could damage a person are included in a range of 30-300 Hz (extremely low frequencies), and 3-30 kHz (very low frequencies). For example, high voltage cables fall within the first range whereas computer screens fall within the second range. Electromagnetic fields emitted by the most part of household appliances have a field of intensity or strength from 3 mG to 1600 mG (60 Hz). As is known by one skilled in the art, an intensity larger than 3 mG would be potentially damaging; accordingly, a safety standard for a total daily exposure to electromagnetic fields should be of 1 mG.

Since our body is accustomed to living immersed in electromagnetic fields, the problem is actually constituted by the fact that the intensity of the fields generated by the above mentioned devices and systems, is much larger than a natural electromagnetic field, and has frequencies near to the exposure frequency; in fact, an electromagnetic field strength will decrease as the distance with respect to the field source increases. Thus, one should hold himself/herself spaced at a safety distance from all the electric sources. However, in most cases, this is not possible; in this connection, consider, for example, those persons who must work throughout the day in front of a computer, and, consequently, very close to a screen different from an LCD screen.

### SUMMARY OF THE INVENTION

The inventor has surprisingly found an efficient remedy for treating and/or preventing symptoms caused by electromagnetic pollution.

Accordingly, the invention specifically relates to the use of natural rubber for making articles or article portions which, in use, are arranged between the human body and ground, in order to prevent and/or treat symptoms caused by electromagnetic field exposure such as sleep disorders, weariness, difficult concentration, irritability, anxiety, memory disorders, cephalea associated with a flushing of the face, female hormonal disorders such as amenorrhea, skin symptoms such as face smarting, itching, acne eruptions, redness, erythema, muscle and joint pain, eye burning, palpitations, tendinitis.

Preferably, the present invention provides to use a natural rubber in a rate of 100%. Preferably, said articles are selected from footwear, footwear soles, seats, desks, beds or any other type of furniture pieces.

More preferably, said articles comprise soles or footwear having natural rubber soles.

The expression “article parts” is meant to indicate inserts, castors, coatings, layers and so on the shape and size of which will depend on the articles they are associated with. Said articles can be made by any prior making methods, which would be well known to one skilled in the art.

The reason for which natural rubber can be used for treating and preventing symptoms caused by electromagnetic pollution, has not yet been clarified. The inventor has surprisingly found that a same positive effect cannot be detected if the mentioned articles of manufacture are made starting from synthetic rubber or leather.

As known, the most common natural rubber materials comprise gum elastic, balata and guttapercha, which polymers are made in the form of a water dispersion (latex), obtained from about 200 species of plants of tropical and subtropical regions. In addition to the polymeric material, said latex comprises different other substances either dissolved or suspended in water such as: enzymes (peroxidasis, catalase, tyrosinase), numerous bacteria, inorganic substances (in particular potassium phosphate), sugars, sterols, fats, and so on.

The polymer particles are of a substantially spherical configuration and have a diameter from few tens and over 2000 nm. They are coated by a protein protective sheath providing the particles with a negative sign charge and, accordingly, then reject one from the other until a coagulation is obtained, usually by adding an acid. This coagulum or clot is separated from serum in the form of a smooth continuous strip and sent to milling apparatus at the output of which the polymer is cut into sheets which, in a layer overlapping relationship, are subjected to fumes to impregnate them with phenolic substances, operating as antiosxidating and antiseptic agents. Thus, that type of commercial gum elastic called “smoked sheet” is made. Alternatively, the sheet elements are caused to pass through cylinders provided with projecting portions and impellers having different angular speed, so as to be stretched and etched. This is performed under a strong current of water to purify the sheets; thus, it is not necessary to perform any smoking operation for preserving the sheet elements. In this manner that type of commercial gum elastic called “crepe” is made.

The average composition of natural rough rubber, independently from the performed processing, can be indicated as follows:

| | | |
|---|---|---|
| polymer | 92-94% | by weight |
| proteic substances | 2-3% | by weight |
| acetonic extract (fat acids, fat acid | 2-4% | by weight |
| esters, sterols, quebracitol and other | | |
| sugars) | | |
| inorganic salts and phosphoric derivatives | 0.5% | by weight |
| moisture | 0.5% | by weight |

The non hydrocarbon substances have very important functions, such as that of stabilizing and protecting the hydrocarbon against oxidation, due to the nitrogen and proteic substances, or to aid the curing, under the effect of the fat acids. The backbone polymer is high steric purity polyisoprene, comprising isoprene monomeric units linked in the 1.4-cis (gum elastic) form. Gutta-percha and balata have a like composition, but the polyisoprene monomeric units have a 1.4-trans pattern, which provides them with less resilient properties. Synthetic rubbers do not comprise polyisoprene, but are made by polymerizing, under the effect of a suitably catalyst, other hydrocarbons such as: butadiene, styrene, vinylbenzene, acrylnitrile, chlorobutadiene and so on. Usually, synthetic rubbers do not have a high steric purity and do not contain all the non hydrocarbon substances included in gum elastic. Independently from any particular theory, the above mentioned substantial differences could represent the reason due to which the above mentioned natural rubber articles provide the above disclosed treating and preventing results, which cannot be found if articles made of synthetic rubber or leather are used.

The present invention will be further herein below disclosed by way of the following not limitative example.

### EXAMPLE

A sample of 30 patients (18 male and 12 female patients) of age from 25 to 65 years, affected by headaches, dizziness and fatigue caused by electromagnetic pollution, was divided into two groups, each made up of 15 people.

A group of said patients were induced to wear footwear having natural rubber soles for three months, whereas a second group was induced to wear, for the same time period, footwear with a synthetic or leather sole.

At the end of the three months period, said 30 patients were subjected to a medical test. Twelve of the fifteen patients, included in the group wearing natural rubber sole footwear, showed a full regression of the symptoms they had at the start of the test; three of said patients registered an improvement in their symptoms corresponding to 50%.

With respect to the fifteen patients included in the group of patients wearing the synthetic or leather sole footwear, the diagnosis was a total persistence of the pathologic symptoms the patient had before the test started.

1. A use of natural rubber for making articles of manufacture or parts of articles of manufacture which, in an use condition, are arranged between the human body and the ground, in order to prevent and/or treat symptoms caused by electromagnetic pollution.

2. A use according to claims 1, wherein said symptoms are selected among: sleep disorders, weariness, difficult concentration, irritability, anxiety, memory disorders, cephalea associated with a flushing of the face, female hormonal disorders such as amenorrhea, skin symptoms such as face smarting, itching, acne eruptions, redness, erythema, muscle and joint pain, eye burning, palpitations, tendonitis.

3. A use according to claim 1, wherein said natural rubber is a 100% natural rubber.

4. A use according to claim 1, wherein said articles comprise footwear, footwear soles, seats, desks or beds.

5. A use according to claim 1, wherein said articles comprise natural rubber soles or footwear having natural rubber soles.

6. A use according to claim 1, wherein said article portions are inserts, coatings, layers or wheels.

7. A use according to claim 1, wherein said article portions have a shape and size varying depending on the article they are associated with.
